(19) United States
(12) Patent Application Publication
(10) Pub. No.: US 2013/0133663 A1
(43) Pub. Date: May 30, 2013

(54) Respiratory Device Filter

(71) Applicants: Geoffrey N. Maksym, Dartmouth (CA); Ewan Kearney MacKenzie, Quebec City (CA); Christopher Gordon Estkowski, Pullman, MI (US); Guy Drapeau, Halifax (CA)

(72) Inventors: Geoffrey N. Maksym, Dartmouth (CA); Ewan Kearney MacKenzie, Quebec City (CA); Christopher Gordon Estkowski, Pullman, MI (US); Guy Drapeau, Halifax (CA)

(73) Assignee: DALHOUSIE UNIVERSITY, Halifax, NS (CA)

(21) Appl. No.: 13/697,235

(22) PCT Filed: May 11, 2011
(86) PCT No.: PCT/US2011/036026

§ 371 Date: Feb. 12, 2013

Related U.S. Application Data
(60) Provisional application No. 61/333,473, filed on May 11, 2010.

(51) Int. Cl. A62B 23/00; A61M 16/04; A61M 16/00; A61B 5/087; A61B 5/097

(52) U.S. Cl. CPC A62B 23/00; A61B 5/087; A61B 5/097; A61M 16/0057; A61M 16/0488
USPC 128/205.27; 29/428

(57) ABSTRACT

A respiratory device filter (10) comprises a housing (12) having a mouthpiece (16) and a body (18). The housing (12) defines an inner sidewall (34) and a conical filter (14) is disposed within the housing (12). The conical filter (14) includes a first end that defines an apex (42) and a second end that defines an opening (44). An apex retention member (46) extends from the inner sidewall (34) and is secured to the apex (42) of the conical filter (14).

### BACKGROUND

Filters may be used to protect medical devices and patients from contaminants, e.g., a filter may be deployed between a pulmonary function testing device, such as a spirometer, and a patient. A patient that employs such a device may have a compromised respiratory system or carry infections and it is desirable to substantially prevent the passage of infectious substances such as viruses or bacteria into that device to avoid contaminating the device and the cross-contamination of subsequent patients who may require the device. Accordingly, placing a filter between the device and a patient facilitates such prevention.

To this end, various filters have been disclosed including those prior art filters discussed in U.S. Pat. No. 5,655,526, U.S. Pat. No. 6,010,458 and U.S. Pat. No. 6,860,526. As provided in the foregoing patents and as is appreciated in the relevant art, it is acknowledged that any such filter should minimize resistance to air flow and minimize the volume of air within the filter that may be rebreathed by the patient known as dead space, as well as prevent bacteria and viruses from passing into the device. Too much dead space within a filter can compromise test results because any re-breathing of gas previously expelled into the filter can cause an undesirable increase in ventilation, placing a higher load on the patient and obtaining test results in an atypical breathing pattern of the patient.

Leakage of air from an incomplete seal between the patient's mouth and the mouthpiece of a filter can compromise test results through incomplete measurement of air flow and/or pressure.

Some pulmonary function testing devices use oscillating fluid flows. During the flow reversal of such an oscillating flow, a filter media can distort its shape, compromising test results, or even reverse itself and enter the patient's mouth thereby causing discomfort or harm to the patient and compromising test results.

The inventors hereof have discovered a filter that has a low resistance to air flow, minimizes dead space and provides protection from microorganisms, including, bacteria and viruses and maintains its shape during flow reversals.

### SUMMARY

Respiratory device filters and methods of manufacturing respiratory device filters are disclosed.

In an implementation, a respiratory device filter comprises a housing having a mouthpiece and a body. The housing defines an inner sidewall and a conical filter is disposed within the housing. The conical filter includes a first end that defines an apex and a second end that defines an opening. An apex retention member extends from the inner sidewall and is secured to the apex of the conical filter.

In an implementation, a method of manufacturing a respiratory device filter comprises the steps of:

forming the conical filter element from a sheet of filter media, the conical filter element having an apex on a first end and defining an opening about a second end;

defining a flange that surrounds the opening of the conical filter element, the flange extending outward and folding over towards the apex of the filter element;

arranging the body of the housing around the conical filter element; securing the apex of the filter element to an apex retention member that extends from an inner sidewall of the body; and arranging and securing the mouthpiece to the body such that at least a portion of the flange is nested in a junction between the mouthpiece and the body such that the opening of the conical filter is coincident with the junction.

In another implementation, a method for manufacturing a filter comprises the step arranging and securing the mouthpiece to the body such that at least a portion of the flange is nested in a junction between the mouthpiece and the body such that the opening of the conical filter is coincident with the junction and secured by it such that tension is imparted to the conical filter.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 provides a perspective view of an exemplary filter;

FIG. 2 provides a perspective, cross-sectional view of the filter housing from the exemplary filter depicted in FIG. 1, taken along the line 2-2;

FIG. 3 provides a perspective, cross-sectional view of the exemplary filter depicted in FIG. 1, taken along the line 2-2;

FIG. 4 provides an detailed view of a portion of the exemplary filter depicted in FIG. 3;

FIG. 5 provides a plan, cross-sectional view of an exemplary filter; and

FIG. 6 provides a graphical representation that depicts some performance characteristics of filters that employ one or more of the features disclosed herein as compared to commercially available filters.

Like reference symbols in the various drawings indicate like elements.

### DETAILED DESCRIPTION

This disclosure provides a respiratory device filter (hereinafter “filter”) that has a low resistance to air flow, a low volume of dead space and removes contaminants such as bacteria and viruses from fluid as it passes through the filter. The disclosed filter is suitable for use in pulmonary function testing devices, such as spirometers, ventilators, forced oscillations devices and mechanical ventilation devices used in intensive care units, operating theaters, neonatal units, and recovery wards among others. In an implementation, the filter is to be located between the testing device and a patient's mouth. The patient breathes into the device via the filter and the filter removes undesirable contaminants. It is to be appreciated, however, that the filter disclosed herein could be used in other applications and the invention hereof should not be limited to the disclosed applications unless specifically required by the claims following this disclosure.

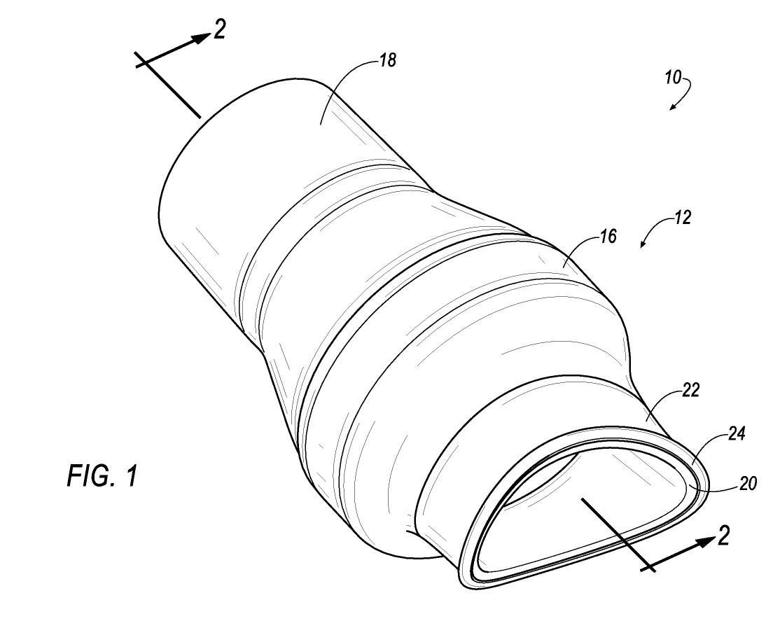

Referring to FIGS. 1-5, an exemplary filter 10 is depicted. Filter 10 includes a housing 12 and a filter element 14 situated within housing 12. Housing 12 comprises a mouthpiece 16 and a body 18.

As provided in FIGS. 1-3 and 5, in an implementation, a first end 20 of mouthpiece 16 may be crescent shaped to allow a patient to seal their lips around first end 20 to simulate a normal breathing position. This construction facilitates sealing between the lips and the filter as the crescent shape strongly correlates to mouth geometry especially at the corners of the mouth, where sealing problems often arise. In an implementation, a bite guard 22 may be placed over first end 20 of mouthpiece 16. As depicted, bite guard 22 may include a bead 24 or similar detent to prevent filter 10 from slipping out of a patient's mouth in a manner that could potentially invalidate testing, injure the patient or the device, or the like. In an implementation, bite guard 22 is overmolded onto first end 20 of mouthpiece 16 and may comprise an elastomeric material. This design can provide a soft positive gripping surface for teeth, create an improved sealing surface and texture for patient lip interface and increase overall patient comfort when using the device. In an implementation, an upper portion of first end 20 of mouthpiece 16 may be substantially flat.

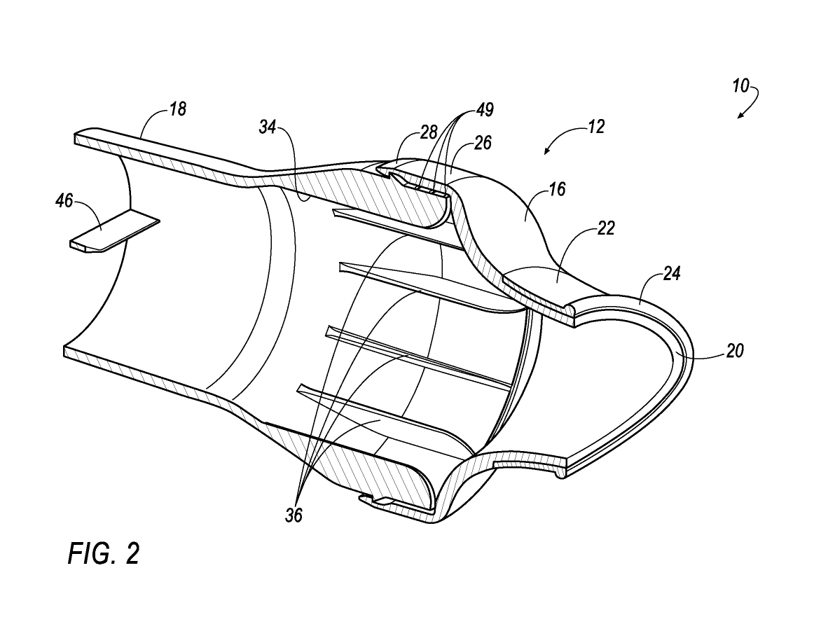

FIGS. 2-5 depict that a second end 26 of mouthpiece 16 is attached to body 18. In an implementation, second end 26 of mouthpiece 16 includes a locking tab 28 and a corresponding, mating portion 30 of body 18 includes a protrusion 31 and a capture rim 32. When second end 26 of mouthpiece 16 and mating portion 30 of body 18 engage, locking tab 28 is sufficiently flexible to slip over protrusion 31 and become seated with capture rim 32 to thereby form an interference connection therebetween.

As shown in FIGS. 1-3 and 5, housing 12 includes an inner sidewall 34. In an implementation, one or more ribs 36 are circumferentially spaced around a portion of inner sidewall 34. Ribs 36 are provided to setoff filter element 14 from inner sidewall 34 and prevent engagement between inner sidewall and filter element 14. This helps to maximize the effective surface area of filter element 14, maintain consistent fluid flow through the filter, maximize useable filtration area, contribute to substantially uniform, circumferential media tensioning without blocking flow, each of which facilitate to improving measurement reproducibility and reliability.

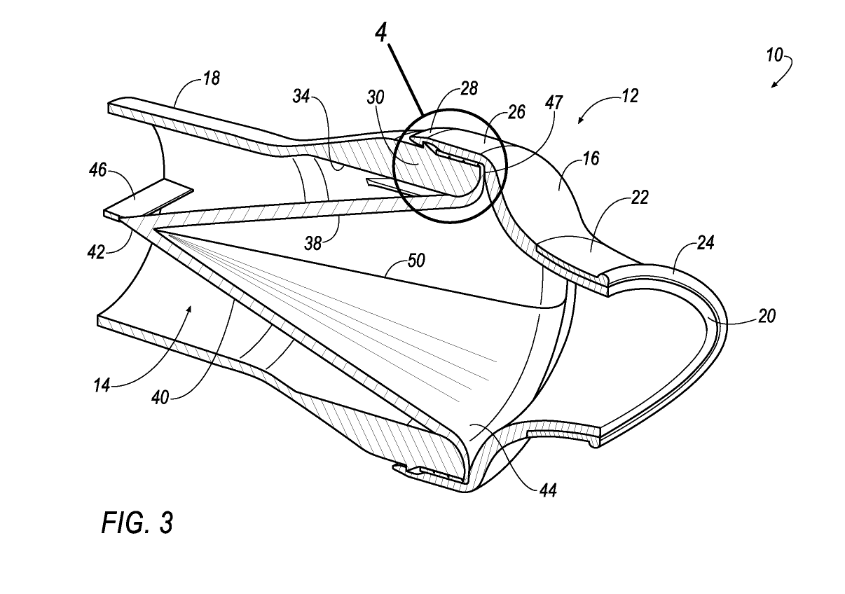

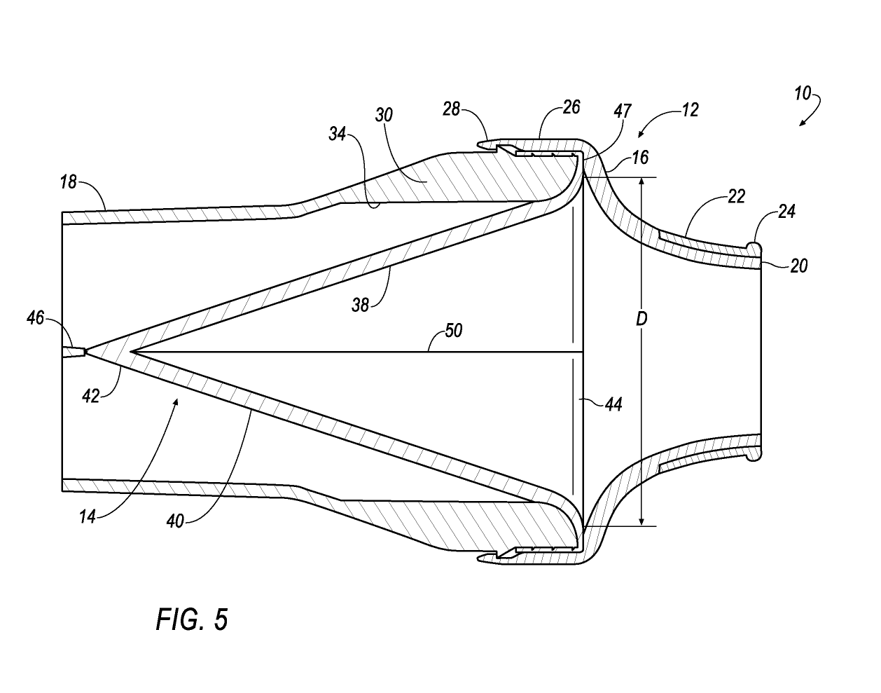

Referring now to FIGS. 3 and 5, and in an implementation, diameter D (or two dimensional area) of the housing 14, as measured across the inner sidewall 34, at the junction between mouthpiece 16 and body 18 is greater than the diameter (or 2-dimensional area) of any other portion within housing 12.

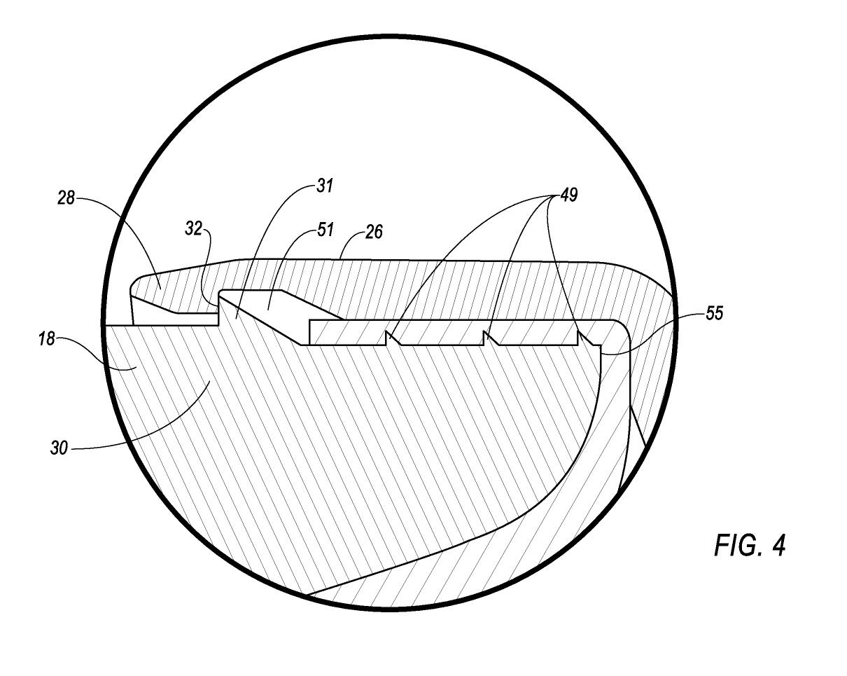

With continued references to FIGS. 3 and 5, filter element 14 is a conical filter and defines an exposed side 38 and a non-exposed side 40. Exposed side 38 faces first end 20 of mouthpiece 16 and non-exposed side 40 opposes exposed side 38. Filter element 14 includes an apex 42 at one end and defines an opening 44 at the other end whereby the media tapers from opening 44 to apex 42. A mounting flange 47 surrounds opening 44 and extends in a direction toward apex 42 and folds or curls outwardly and back towards body 18. In an implementation, mounting flange 47 is nested between mating portion 30 of body 18 and second end 26 of mouthpiece 16. In an implementation, Elating portion 30 of body 18 includes one or more retention members 49 on an outer facing sidewall 53 between protrusion 31 and an end 55 of body 18, as best shown in FIG. 4. Among other things, retention members 49 are provided as gas checks to seal the filter such that fluid passing through filter 10 cannot escape between the junction between mouthpiece 16 and body 18. In an implementation, a well 51 may be provided between retention members 49 and protrusion 31 so that adhesive may be disposed therebetween to adhesively secure mouthpiece 16 to body 18. It is to be understood that the apex of the conical filter may not be a discrete point and is simply used herein to denote an end portion of the conical filter and the invention should not be limited thereby.

In an implementation, opening 44 of filter element is coincident with the junction between mouthpiece 16 arid body 18 such that the maximized housing diameter D (or 2-dimensional cross-sectional area) also corresponds to the diameter of opening 44.

With reference to FIGS. 2, 3 and 5, body 18 includes an apex retention member 46 that interacts with apex 42 to secure apex 42 in a static position. This design prevents filter element 14 from moving, fluttering and inverting leading to risk of swallowing, which can arise during a reversal of fluid flow, among other ways. Apex retention member 46 also tensions the filter to thereby yield increased effective filtration surface area, decreased flow resistance within housing 12 and the like. In an implementation, apex retention member 46 is a cross-member that extends from one portion of the sidewall 34 of housing 12 to another portion of the inner sidewall 34 of housing 12 and is secured to apex 42. Apex retention member 46 includes opposing slanted surfaces that converge at the apex and diverge in the other direction so as to reduce fluidic restriction and provide an aerodynamic design. Apex 42 may be adhesively connected to apex retention member 46. In an implementation, filter element 14 includes a seam 50. In an implementation, seam 50 and apex retention member 46 may be correspondingly aligned.

A method of manufacturing a filter will now be disclosed. In an implementation, filter element 14 is manufactured from a sheet of filter media. The sheet of filter media may be cut and welded in a single operation to form a conical filter media structure having a single seam 50. Filter element 14 is thereafter arranged on an inversion cone to form mounting flange 36 whereby the flange becomes inverted and extends back toward apex 42. Next, body 18 is placed onto the inverting cone with the filter element 14 in place and oriented so that seam 50 is aligned with apex retention member 46. The cone is removed and apex retention member 46 is secured to apex 42 of filter element 14 by way of an adhesive or other securing means. Next, mouthpiece 16 is secured to body 18. In an implementation, an adhesive bead is disposed on one or both of mouthpiece 16 and body 18 proximate to where well 51 will be arranged. Mouthpiece 16 is slid onto body 18 such that locking tab 28 flexes over protrusion 31 until it snaps back into place and seats along the capture rim 32. In an implementation, mouthpiece 16 is oriented with the major axis aligned with the seam of filter element 14. As a result of this process, one or more retention member 49 engage filter media against an inner wall of locking tab 28 to prevent the media from slipping out of position during the manufacturing process. The top rim of the body 18 impinges the filter element 14 at the transition of the flange against the interior wall of the mouthpiece 16 to secure the filter element 14 from loosening during storage and handling.

In an implementation, filter element 14 can be secured both by one or more retention members 49 which prevent the conical filter element 14 from moving excessively during assembly, but also by the compression of the filter element 14 between the top of the body 18 and the underside of the mouthpiece 16. In this implementation, this particular feature requires adjusting the molding tools to produce the two parts with the precise amount of filter media compression and still allow the snap fit geometry to find home position.

As depicted, mouthpiece 16 and body 18 are discrete elements and joined in the manufacturing process, yet it is conceived that mouthpiece 16 and body 18 may be integrally joined. The two-piece design, however, provides manufacturing latitude such that different mouthpieces 16 or bodies 18 may be interchanged as may be needed for variations of respiratory devices, including, pulmonary testing applications or pulmonary or respiratory testing devices.

In an implementation, the adhesive to secure apex retention member 46 to apex 42 and body 18 to mouthpiece 16 is cyanoacrylate, however, it is to be appreciated that a variety of applications may be utilized.

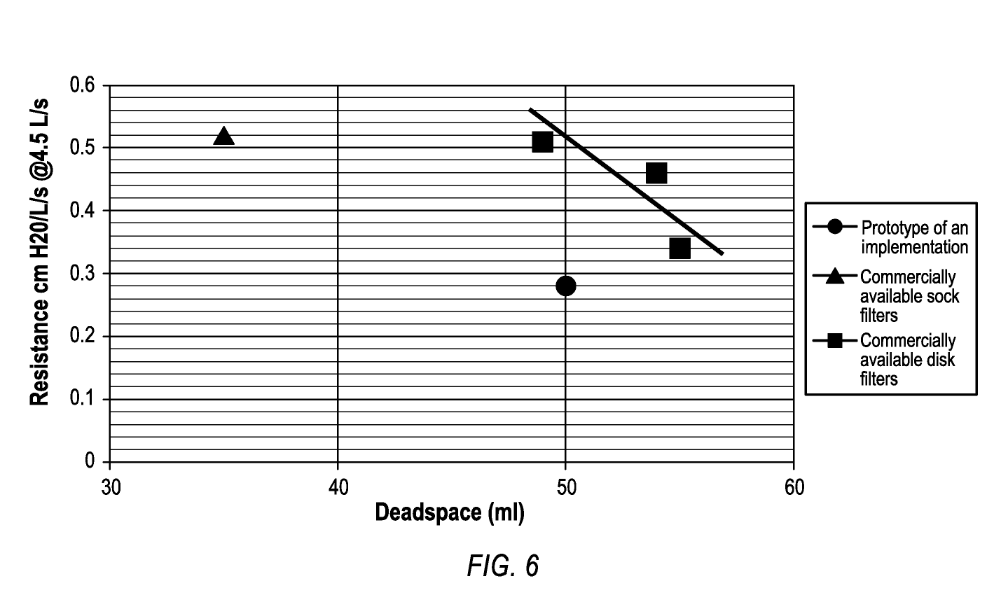

As discussed, it is desirable to minimize both resistance to air flow and dead space at a given filtration efficiency. The inventors hereof have discovered that for a given filtration efficiency, a better compromise between dead space and resistance can be achieved than is available in typical commercial pulmonary function testing filters. FIG. 6 illustrates the performance of an implementation of a prototype of the invention that employs the disclosed features as compared to commercially available disk and sock filters. FIG. 6 shows an optimized balance between resistance and dead space achieved by a prototype of an implementation when compared to commercially available filters. The inventors hereof have discovered that by varying the diameter of the body 18 such that it is largest where the diameter of the conical filter member 14 is largest, and the diameter of the body 18 is reduced towards the apex 42 of the cone in filter element 14, the dead space can be reduced while maintaining low resistance to flow.

The state of third party pulmonary function testing filters, including disk, sock and pleated filters include filters that suffer from either one or both of excessive dead space and too much flow resistance. Pleated filters appear to be the least desirable because they are less efficient than disk filters when they have the same resistance. Disk filters, however, have a high amount of dead space whereas the tested sock filters have sufficient dead space, but high resistance. As shown by FIG. 6, the disclosed filter design improves upon the characteristics known in filter devices currently in commercial production.

The field of spirometer testing device filters have typically provided heavier filter media that yield a higher filtration efficiency but provide the disadvantage of filter resistance which increases linearly as the media weight increases, such that an optimum balance must be struck therebetween.

In an implementation, the filter media can include any material that is commonly used in the manufacture of spirometer or other pulmonary function testing device filters, including without limitation, needle punch felts, electrostatic filter material, including woven or non-woven fibers that may be synthetic or natural materials. In one illustrative implementation, the filter material can include electret filter material or triboelectric filter medium as disclosed in U.S. Pat. No. 6,328,788 incorporated herein in its entirety. Exemplary triboelectric media materials can include that which is sold under the trade name Alphastar, which weighs 60 gsm and is manufactured by Delstar Technologies Inc., 601 Industrial Drive, Middletown, Del. 19709 USA. In an implementation, the filter media may be Texel Tribo 100 HJ, which weighs 100 gsm or the tribo 100 which weighs 115 gsm and has a production range of 95-135 gsm. In some embodiments, the filter element 14 can be properly tensioned thereby influencing the resistance of fluid passing through the filter 10 since the proper tension provides a geometry of filter element 14 that allows the flow of air to uniformly utilize the entire filter element 14, including exposed side 38. This can be accomplished by reducing turbulence within filter element 14 that manifests as resistance. In one implementation, the tensioning process acts upon the filter media without over stressing the media or creating media inefficiencies (voids). The tensioning process can include the steps of capturing the filter media at the weld line (not shown) where it is seamed and cut to size. This area is reinforced by the nature of the cut and weld process to double material thickness compressed via the ultrasonic welding process and melted. This creates a very strong linear “cord” (not shown) that can be tensioned with a considerable amount of force that would normally compromise the media in its original state over long term and result in media matrix voids. The tensioning cord (not shown) has a specific orientation in the filter element 14 to facilitate capturing the end of the cord and bonding it to the apex retention member 46 adequately. The apex 42 of filter element 14 is attached to the apex retention member 46 first then slack in filter element 14 is taken up by properly securing the filter element 14 over body 18 and slidably attaching mouthpiece 16 as described above. When manufactured, orienting the filter element 14 to utilize the strongest direction of fiber further enhances this tensioning technique. In an implementation, filter element 14 cone geometry is slightly tensioned diametrically to create a stable conical shape with uniform structure to minimize turbulent flow. This is initiated at the mating portion 30 of body 18 which includes a protrusion 31 and a capture where the filter element 14 folds over the body 18 and secured by mouthpiece 16. Filter element 14 size controls the tension so an accurate cone shape in filter element 14 can provide proper placement and tensioning.

A number of implementations have been described. Nevertheless, it will be understood that various modifications may be made without departing from the spirit and scope of the disclosure. Accordingly, other implementations are within the scope of the following claims.

What is claimed is:

1. A filter (10) comprising:
a housing (12) having a mouthpiece (16) and a body (18), the housing (12) defining an inner sidewall (34);
a conical filter (14) disposed within the housing (12), the conical filter (14) having a first end that defines an apex (42) and a second end that defines an opening (44); and
an apex retention member (46) extending from the inner sidewall (34) and secured to the apex (42) of the conical filter (14).

2. The filter (10) according to claim 1, wherein the mouthpiece (16) and the body (18) are discrete elements that are securingly attached to one another and form a junction therebetween.

3. The filter (10) according to claim 2, wherein the filter (10) includes a flange (47) that axially extends from the second end of the conical filter (14) toward the apex (42), and wherein the flange (47) is nested in at least a portion of the junction between the mouthpiece (16) and the body (18) such that the opening (44) of the conical filter (14) is coincident with the junction.

4. The filter (10) according to claim 3, wherein the mouthpiece (16) has a first end (20) that defines an opening that is crescent shaped to thereby create a good seal around a user's mouth when in use, and wherein the first end (20) includes a bite guard (22) surrounding the opening having a bead (24) extending therefrom to prevent a patient's mouth from slipping off the first end (20).

5. The filter (10) according to claim 1, wherein the mouthpiece (16) has a second end (26) that includes a flexible locking tab (28), and wherein the body (18) includes a mating portion (30) that defines a protrusion (31) and a capture rim (32), wherein the locking tab (28) nests within the capture rim (32) of the mating portion (30).

6. The filter (10) according to claim 5, wherein the mating portion (30) includes filter element retention members (49) between the protrusion (31) and the end of the mating portion (30), and wherein the filter element retention members (49) compressibly engages the conical filter (14) against the flexible locking tab (28).

7. The filter (10) according to claim 1, wherein the apex retention member (46) includes opposing, sloped surfaces that converge toward the apex (42) to minimize restriction of fluid flow in the direction from the mouthpiece (16) to the body (18).

8. The filter (10) according to claim 1, wherein the inner sidewall (34) along the body (18) includes two or more ribs (36) that extend from the inner sidewall (34) toward the conical filter (14) to setoff the conical filter (14) from the inner sidewall (34).

9. The filter (10) according to claim 8, wherein the two or more ribs (36) are circumferentially arranged around the inner sidewall (34).

10. The filter (10) according to claim 1, wherein the conical filter (14) includes a seam (50), and wherein the seam (50) is axially aligned with a cross-member.

11. The filter (10) according to claim 1, wherein the diameter of the inner sidewall (34) of the housing (12) that is coincident with the opening is greater or equal to any other inner sidewall diameter of the housing (12).

12. A method of manufacturing a filter (10), the filter (10) having a conical filter element (14) disposed within a housing (12), the housing (12) have a mouthpiece (16) and a body (18), the method comprising:
forming the conical filter element (14) from a sheet of filter media, the conical filter element (14) having an apex (42) on a first end and defining an opening (44) about a second end;
defining a flange (47) that surrounds the opening of the conical filter element (14), the flange (47) extending outward and folding over towards the apex (42) of the conical filter element (14);
arranging the body (18) of the housing (12) around the conical filter element (14);
securing the apex (42) of the conical filter element (14) to an apex retention member (46) that extends from an inner sidewall (34) of the body (18); and
arranging and securing the mouthpiece (16) to the body (18) such that at least a portion of the flange (47) is nested in a junction between the mouthpiece (16) and the body (18) such that the opening (44) of the conical filter element (14) is coincident with the junction.

13. The method according to claim 12, wherein the flange (47) is defined by arranging the conical filter element (14) on an inversion cone such that the flange (47) becomes inverted and extends back toward the apex (42).

14. The method according to claim 13, wherein the step of arranging the body (18) further comprises:
placing the body (18) onto the inversion cone with the conical filter element (14) in place and oriented so that a seam (50) of the conical filter element (14) is aligned with the apex retention member (46).

15. The method according to claim 14, wherein the mouthpiece (16) has a crescent shaped first end (20) and a second end that includes a flexible locking tab (28), and wherein the body (18) includes a mating portion that defines a protrusion (31) and a capture rim (32), and further wherein the step of arranging and securing the mouthpiece (16) to the body (18) further comprises:
sliding the mouthpiece (16) onto the body (18) such that the flexible locking tab (28) flexes over the protrusion (31) until it snaps back into place and seats along the capture rim (32).

16. The method according to claim 15, wherein the step of arranging and securing the mouthpiece (16) to the body (18) further comprises:
dispensing an adhesive on one or both of mouthpiece (16) and body (18) proximate to the junction therebetween.

17. The method according to claim 14, wherein the mating portion of the body (18) includes filter element retention members (49) between the protrusion (31) and the end of the mating portion and extending toward an inner sidewall of the flexible locking tab (28) of the mouthpiece (16), and wherein the step of arranging and securing the mouthpiece (16) to the body (18) further comprises:
compressibly engaging the conical filter element (14) between the filter element retention members (49) and the inner sidewall of the flexible locking tab (28).

18. The method according to claim 15, wherein the step of arranging and securing the mouthpiece (16) to the body (18) further comprises:
orientating the mouthpiece (16) so that its major axis is axially aligned with the seam (50) of the conical filter element (14).

19. The method according to claim 12, wherein the conical filter element (14) includes a seam (50), the method further comprising:
orienting one or both of the conical filter element (14) or the body (18) so that the seam (50) is axially aligned with the apex retention member (46).

20. The method according to claim 12, further comprising:
overmolding an elastomeric bite guard (22) on a first end (20) of the mouthpiece (16) and defining a circumferential bead thereon.
